(19) United States
(12) Patent Application Publication
(10) Pub. No.: US 2019/0313955 A1
(43) Pub. Date: Oct. 17, 2019

(54) MULTISCALE MICRODEVICES WITH NANOPILLARS FOR CHRONICALLY IMPLANTED DEVICES

(71) Applicant: Purdue Research Foundation, West Lafayette, IN (US)

(72) Inventor: Hyowon Lee, West Lafayette, IN (US)

(73) Assignee: Purdue Research Foundation, West Lafayette, IN (US)

(21) Appl. No.: 16/379,743

(22) Filed: Apr. 9, 2019

Related U.S. Application Data
(60) Provisional application No. 62/657,028, filed on Apr. 13, 2018.

(51) Int. Cl. A61B 5/1486; B81C 1/00; B82Y 15/00; B81B 7/02

(52) U.S. Cl. CPC A61B 5/14865; B81C 1/00031; B81B 2203/0361; B81B 7/02; B81B 2201/0214; B82Y 15/00

(57) ABSTRACT

Disclosed herein is an antifouling device for large, nonplanar optical surfaces. The device can be used as marine or implantable applications that has nested multiscale features for active and passive anti-biofouling. By combining active and passive anti-biofouling mechanisms, this device will provide long-term protection against biofouling for over 10 years, extending the lifetime of self-clearing marine or implantable sensors.

### CROSS REFERENCE

This application claims the benefits of U.S. provisional application 62/657,028, filed on Apr. 13, 2018. The content of which is expressly incorporated herein entirely.

### FIELD OF INVENTION

This disclosure relates to an implantable device with multiscale morphology to enhance its functional longevity. Particularly, microscale needle arrays with biomimetic nanopillars are used in combination with active actuators for combating multiscale biofouling in implantable biosensors and drug delivery devices.

### BACKGROUND

Biofouling is one of the most recognized challenges in developing chronically functional (for decades) devices for marine and implantable applications. Microscale sensors and drug delivery devices often suffer from functional degradation due to biofouling. For example, the state-of-the-art continuous glucose monitoring implants have approved lifetime of less than 90 days. Moreover, the US Navy spends >$400 million annually to maintain its fleet against biofouling. This problem has been addressed by the use of coating; however, coatings lose functionality over time so it is short-term solution. Passive antifouling mechanisms (e.g., materials, surface morphology, sacrificial layers) are often toxic, short-term solutions that eventually succumb to complex multiscale (i.e., molecular, cellular) biofouling. Active mechanisms (e.g., UV, mechanical, electrical) are better for long-term management, but they are ill-suited for large nonplanar surface due to mechanical impedance mismatch and power requirement.

A more reliable and long-lasting solution might improve the reliability and performance of implantable sensors and drug delivery devices.

### SUMMARY OF THE INVENTION

This disclosure provides an antifouling solution for large, nonplanar surfaces. The disclosed device for marine or implantable applications uses nested multiscale features for active and passive anti-biofouling strategies. By combining active and passive anti-biofouling mechanisms, this device will provide long-term protection against biofouling for over 10 years, extending the lifetime of self-clearing marine or implantable sensors.

The advantages of such device is to remove protein, bacteria, and cellular-level biofilms, to provide extend lifetime of self-clearing marine/implantable sensors and to provide insights on progression of various chronic diseases.

With proper morphology and material design, the multiscale micro devices with nanopillars may be used in various applications including but not limited to marine/implantable sensors, neural interfaces, inflammation, restoration, and therapeutics.

These and other features, aspects and advantages of the present invention will become better understood with reference to the following figures, associated descriptions and claims.

### BRIEF DESCRIPTION OF THE DRAWINGS

FIGS. 1A-1B Top view and sectional views of a biosensor with nested micropillars and nanopillars configured on sensor electrodes sitting on a magnetic element.

FIG. 2 A proposed plot of sensor sensitivity versus implantation time of a nested biosensor with or without active actuation.

### DETAILED DESCRIPTION

While the concepts of the present disclosure are illustrated and described in detail in the figures and the description herein, results in the figures and their description are to be considered as exemplary and not restrictive in character; it being understood that only the illustrative embodiments are shown and described and that all changes and modifications that come within the spirit of the disclosure are desired to be protected.

Unless defined otherwise, the scientific and technology nomenclatures have the same meaning as commonly understood by a person in the ordinary skill in the art pertaining to this disclosure.

To address key challenges such as multiscale biofouling on large surface, we fabricate biomimetic nanopillars on polymeric microposts using nanosphere lithography to create multiscale nested morphology with passive antibacterial functionality, and integrate biomimetic nanopillars on magnetic microactuator arrays for active removal of proteins, bacteria and other cellular biofilm.

To verify perpetual multiscale antibiofouling properties, we develop accelerated biofouling evaluation platforms using proteinaceous (bovine serum albumen), bacterial (fluorescent E-coli), and cellular (ECM-enhanced 3D culture) biofilms, and measure impact of actuation amplitude, frequency, and duty cycle on biofilm removal.

To ensure chronic sensor longevity against biofouling, we integrate device with optical oxygen sensor and quantify optical attenuation; and verify minimum longitudinal changes in sensitivity, range, linearity, limit of detection.

Methods and Approach We will develop novel polymer-based multi-scale magnetic actuator arrays with nested nanopillars. The scalable polymeric thin-film device with high mechanical compliance facilitates integration with large nonplanar surfaces without significant functional attenuation. The biomimetic nanopillars feature antibacterial properties via penetration and low surface energy. The magnetic actuation, which requires zero on-chip power, can generate large forces to actively remove protein (>69%), bacteria, and cellular biofilm (>48%)

### EXAMPLE 1

### Drug Elution/Long Term Drug Delivery and Biosensing

To enable high-fidelity, longitudinal biomarker monitoring and long-term drug delivery, strategies for combatting multiscale biofouling-related failure modes for minimally invasive drug delivery devices and biosensors are needed. By combining active and passive mechanisms of anti-biofouling approaches, the novel designed micro device has capability of increasing the functional lifetime of minimally invasive drug delivery needles and continuous glucose monitoring devices from days to years. These new features are critical in developing closed-loop therapeutics for various chronic illnesses including diabetes and neurological disorders.

In developing multiscale drug delivery device, we create microscale needle arrays with biomimetic nanopillars using 3D nanosphere lithography. In some embodiment, the microneedle arrays are functionalized with anti-inflammatory biocompatible coatings including type I collagen, chitosan, or zwitterionic polymers.

One embodiment is to develop an electrochemical glucose biosensors on active and passive anti-biofouling actuation platforms (i.e., magnetically or ultrasound powered).

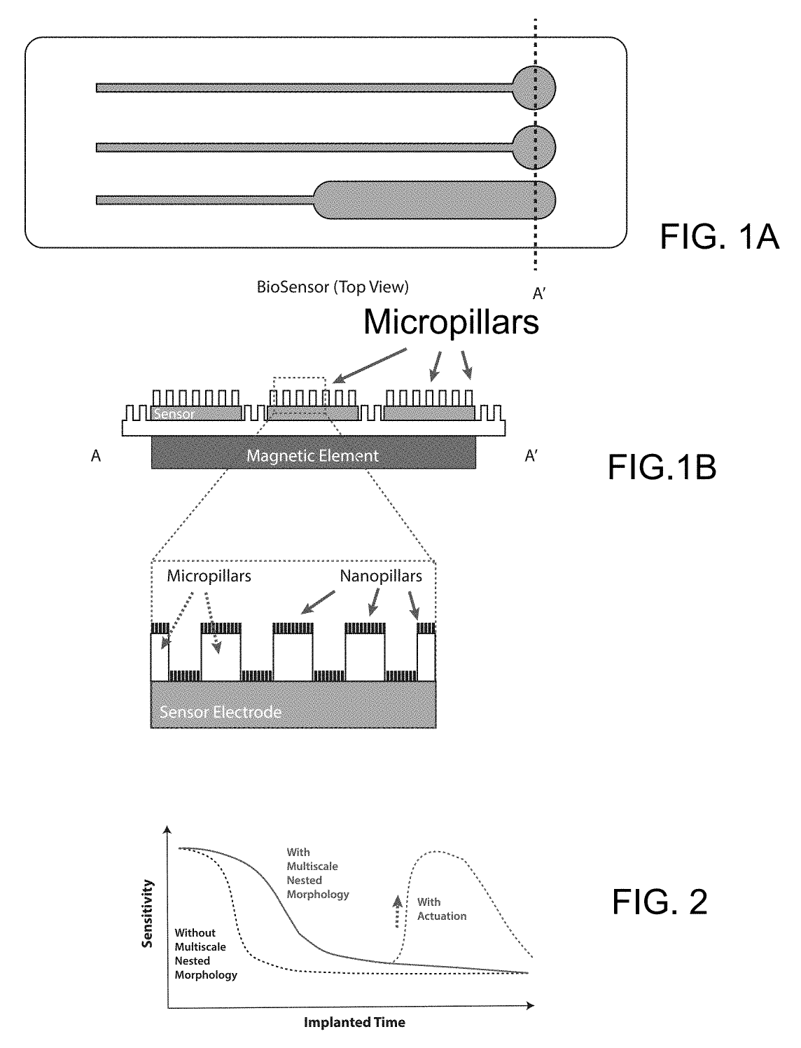

One example of such combined actuator is shown in FIG. 1, wherein a nested nanopillar microneedles are configured on top of and in between a plurality of micropillars, with the latter sitting on or in between sensor electrodes and can be actuated by magnetic elements.

It is important to integrate active and passive anti-biofouling strategies for drug delivery application so that a prolonged sensitivity plot versus implantation time as shown in FIG. 2 is achieved.

It is within the skill of art based on above description to fabricate a prototype of multiscale anti-biofouling microneedles with optimum biomimetic nanopillar design, as shown in FIGS. 1A and 1B.

Following the design and prototype production of multiscale anti-biofouling, it is important to compare the performance of nanopillars with or without anti-inflammatory polymer coating, active actuation individually and in combination.

When successful, this technology will provide unprecedented insights on the progression of various chronic diseases (e.g., diabetes, neurological disorders) by allowing longitudinal high-fidelity monitoring of patients. Broadly, this development will significantly push the boundary at the forefront of advanced materials, maritime technology, sensors, neural interface, inflammation, restoration, and therapeutics.

1. A biosensor comprising a polymer-based multi-scale active actuator consisting of arrays with nested nanopillars configured on/and between a plurality of micropillars.

2. The biosensor according to claim 1, wherein the nested nanopillars and micropillars are coated with anti-inflammatory biocompatible materials selected from the group consisting of type I collagen, chitosan, and zwitterionic polymers.

3. The bio sensor of claim 1 is used for glucose monitoring in diabetes conditions.

4. The bio sensor of claim 1 is for measurement of neurological disorders.

5. The biosensor of claim 1 wherein the nested nanopillars consisting of 3D nanosphere lithography created microneedles.

6. The biosensor according to claim 1 wherein the multiscale active actuator is powered by magnetic or ultrasound.

7. A method of prolonging the use life of an implantable biosensor or drug delivery device in a patient, comprising:
Providing to the patient a polymer-based multi-scale active actuator consisting of arrays with nested nanopillars configured on/and between a plurality of micropillars, wherein said arrays are optionally coated with anti-inflammatory biocompatible materials and are powered by magnetic or ultrasounds;
Recording the sensitivity of the biosensor or the kinetics of drug delivery device in the patient versus the implantation time;
Identifying a time point of the sensitivity or the kinetics of drug delivery device reaching a bottom plateau without actuation; and
activating the actuator until the sensitivity or the kinetics of drug delivery device reaching the top plateau.

8. The method according to claim 7, wherein the bio sensor is a glucose monitoring device.

9. The method according to claim 7 wherein the nested nanopillars consisting of 3D nanosphere lithography created microneedles.
